(19) United States
(12) Patent Application Publication
(10) Pub. No.: US 2024/0199784 A1
(43) Pub. Date: Jun. 20, 2024

(54) LATEX COMPOSITION FOR DIP-FORMING, METHOD FOR PREPARING THE SAME, AND DIP-FORMED ARTICLE FORMED BY THE SAME

(71) Applicant: KOREA KUMHO PETROCHEMICAL CO, LTD., Seoul (KR)

(72) Inventors: Sung Hun CHOUNG, Daejeon (KR); Ha Jung LEE, Seoul (KR); Jeong Weon HAN, Daejeon (KR)

(73) Assignee: KOREA KUMHO PETROCHEMICAL CO, LTD., Seoul (KR)

(21) Appl. No.: 18/526,222

(22) Filed: Dec. 1, 2023

(30) Foreign Application Priority Data
Dec. 15, 2022 (KR) 10-2022-0175910

(51) Int. Cl. C08F 236/08; A41D 19/00; C08F 2/38; C08F 2/60

(52) U.S. Cl. CPC C08F 236/08; A41D 19/0055; C08F 2/38; C08F 2/60; C08F 2400/04

(57) ABSTRACT

One embodiment of the present specification provides a method for preparing a latex composition for dip-forming, the method including: (a) preparing a monomer mixture including a conjugated diene-based monomer, a farnesene monomer, an ethylenically unsaturated nitrile monomer, and an ethylenically unsaturated acid monomer; (b) adding an additive including an emulsifier and an inorganic solvent to the monomer mixture; and (c) polymerizing the monomer mixture to prepare a copolymer latex, wherein a weight ratio of the farnesene monomer to a total weight of the conjugated diene-based monomer and the farnesene monomer is 5 to 55%.

### CROSS-REFERENCE TO RELATED APPLICATION

This application claims priority to and the benefit of Korean Patent Application No. 2022-0175910, filed on Dec. 15, 2022, the disclosure of which is incorporated herein by reference in its entirety.

### BACKGROUND

### 1. Field of the Invention

The present specification relates to a latex composition for dip-forming, a method for preparing the same, and a dip-formed formed by the same.

### 2. Discussion of Related Art

Conventionally, the main raw material for gloves used for medical purposes, agricultural and livestock product processing, or industrial purposes was natural rubber latex. However, when gloves made from natural rubber latex are used, the problem of the glove user suffering from a contact allergic disease due to the proteins contained in natural rubber latex frequently occurs. Accordingly, attempts have been made to manufacture gloves by applying synthetic rubber latex that does not contain protein, such as nitrile-based copolymer latex. Nitrile-based copolymer latex gloves have superior mechanical strength compared to natural rubber latex gloves, and thus the demand therefor is increasing in medical and food fields where contact with sharp objects occurs frequently.

However, it is difficult for existing nitrile-based copolymer latex gloves to naturally decompose, causing serious environmental pollution problems due to the waste disposal thereof. Accordingly, there is an increasing social demand for developing nitrile-based copolymer latex gloves that satisfy mechanical properties, such as tensile strength and elongation, and durability that are required for latex gloves, while using environment-friendly and renewable materials.

In addition, existing nitrile-based copolymer latex gloves have the problem of inner surfaces adhering to each other or stickiness occurring in the process of inserting the hand into the glove. When powder treatment is performed in the post-processing process to improve this, a problem occurs that the powder gets on the hands or causes contamination in an operating room. Therefore, there is a need for developing nitrile-based copolymer latex gloves that have excellent mechanical properties and durability and improve the stickiness inside the gloves.

### SUMMARY OF THE INVENTION

The description in the present specification is intended to solve the problems of the related technology described above, and one object of the present specification is to provide a method for preparing a latex composition for dip-forming that is environment-friendly and exhibits excellent mechanical stability.

Another object of the present specification is to provide a latex composition for dip-forming that has excellent mechanical properties, such as tensile strength and elongation, and durability and improved stickiness; and a dip-formed prepared therefrom.

According to one aspect, provided is a method for preparing a latex composition for dip-forming, the method including: (a) preparing a monomer mixture including a conjugated diene-based monomer, a farnesene monomer, an ethylenically unsaturated nitrile monomer, and an ethylenically unsaturated acid monomer; (b) adding an additive including an emulsifier and an inorganic solvent to the monomer mixture; and (c) polymerizing the monomer mixture to prepare a copolymer latex, wherein a weight ratio of the farnesene monomer to a total weight of the conjugated diene-based monomer and the farnesene monomer is 5 to 55%.

In one embodiment, the conjugated diene-based monomer may be one selected from the group consisting of 1,3-butadiene, 2,3-dimethyl-1,3-butadiene, 2-ethyl-1,3-butadiene, 1,3-pentadiene, isoprene, and a combination of two or more thereof.

In one embodiment, the farnesene monomer may be an α-farnesene monomer, a β-farnesene monomer, or a combination thereof.

In one embodiment, the ethylenically unsaturated nitrile monomer may be one selected from the group consisting of acrylonitrile, methacrylonitrile, fumaronitrile, α-chloroacrylonitrile, α-cyano ethyl acrylonitrile, and a combination of two or more thereof.

In one embodiment, the ethylenically unsaturated acid monomer may be one selected from the group consisting of methacrylic acid, acrylic acid, itaconic acid, maleic acid, fumaric acid, maleic anhydride, citraconic anhydride, styrene sulfonate, monobutyl fumarate, monobutyl maleate, mono-2-hydroxypropyl maleate, and a combination of two or more thereof.

In one embodiment, the additive may further include a molecular weight regulator.

In one embodiment, the molecular weight regulator may be one selected from the group consisting of mercaptans such as an α-methylstyrene dimer, t-dodecyl mercaptan, n-dodecyl mercaptan, and octyl mercaptan; halogenated hydrocarbons such as carbon tetrachloride, methylene chloride, and methylene bromide; sulfur-containing compounds such as tetraethyl thiuram disulfide, dipentamethylene thiuram disulfide, and diisopropyl xanthogen disulfide; and a combination of two or more thereof.

In one embodiment, a content of the molecular weight regulator may be 0.2 to 0.65 parts by weight based on 100 parts by weight of the monomer mixture.

In one embodiment, a gel content of the copolymer latex prepared in (c) above may be 80% or less.

According to another aspect, provided is a latex composition for dip-forming, prepared by the above preparation method.

According to still another aspect, provided is a dip-formed article formed by dip-forming the latex composition for dip-forming.

In one embodiment, the dip-formed article may be one selected from the group consisting of medical gloves, gloves for agricultural and livestock product processing, and industrial gloves.

### DETAILED DESCRIPTION OF EXEMPLARY EMBODIMENTS

Hereinafter, one aspect of the present specification will be described with reference to an embodiment. However, the description of the present specification may be implemented in various different forms, and thus is not limited to the embodiments described herein. In addition, in order to clearly explain each content for the implementation of the present specification, parts not related to the description or widely known in the art have been omitted, and similar parts are given similar reference numerals throughout the specification.

Throughout the specification, when a part is “connected” to another part, this not only includes the case where it is “directly connected” but also the case where it is “indirectly connected” with another member interposed therebetween. In addition, when a part is said to “include” a component, this means that other components may be further included, not excluded, unless specifically stated otherwise.

When a range of numerical values is recited herein, the values have the precision of the significant figures provided in accordance with the standard rules in chemistry for significant figures, unless a specific range is otherwise stated. For example, 10 includes the range of 5.0 to 14.9, and 10.0 includes the range of 9.50 to 10.49.

Hereinafter, embodiments of the present specification will be described in detail.

### Method for Preparing Latex Composition for Dip-Forming

A method for preparing a latex composition for dip-forming according to one aspect of the present specification includes: (a) preparing a monomer mixture including a conjugated diene-based monomer, a farnesene monomer, an ethylenically unsaturated nitrile monomer, and an ethylenically unsaturated acid monomer; (b) adding an additive including an emulsifier and an inorganic solvent to the monomer mixture; and (c) polymerizing the monomer mixture to prepare a copolymer latex.

The weight ratio of the farnesene monomer to the total weight of the conjugated diene-based monomer and the farnesene monomer may be 5 to 55%. For example, the weight ratio of the farnesene monomer to the total weight of the conjugated diene-based monomer and the farnesene monomer may be 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, or a value between two of these values. When the weight ratio of the farnesene monomer to the total weight of the conjugated diene-based monomer and the farnesene monomer is less than the range described above, the mechanical stability of the latex may decrease or the stickiness of the final formed product may increase, and the durability thereof may decrease; and when the range described above is exceeded, a latex polymerization reaction rate may decrease, the gel content of latex may excessively increase, or the tensile strength and elongation of the final formed product may decrease. For example, when the weight ratio of the farnesene monomer to the total weight of the conjugated diene-based monomer and the farnesene monomer exceeds the range described above, the gel content of latex may exceed 80%, or the tensile strength and elongation of the final formed product may decrease to less than 90% of a level of an article formed of an existing latex composition for dip-forming that does not include a farnesene monomer, and thus the mechanical properties required for latex gloves may not be satisfied.

Step (a) above is a step of preparing a monomer mixture including a conjugated diene-based monomer, a farnesene monomer, an ethylenically unsaturated nitrile monomer, and an ethylenically unsaturated acid monomer, which constitute a copolymer, and may be performed under a nitrogen atmosphere.

The conjugated diene-based monomer may be one selected from the group consisting of 1,3-butadiene, 2,3-dimethyl-1,3-butadiene, 2-ethyl-1,3-butadiene, 1,3-pentadiene, isoprene, and a combination of two or more thereof, and for example, it may be 1,3-butadiene, but is not limited thereto.

The content of the conjugated diene-based monomer may be 40 to 70% by weight based on the total weight of the monomer mixture. For example, it may be 40% by weight, 45% by weight, 50% by weight, 55% by weight, 60% by weight, 65% by weight, 70% by weight, or a value between two of these values. When the content of the conjugated diene-based monomer is less than the range described above, the gel content of latex may excessively increase or the tensile strength and elongation of the final formed article may decrease; and when the range described above is exceeded, the mechanical stability of latex may decrease or the stickiness of the final formed article may decrease, and the durability thereof may decrease.

The farnesene monomer may be an α-farnesene monomer, a β-farnesene monomer, or a combination thereof, and for example, it may be a β-farnesene monomer, but is not limited thereto. The farnesene monomer may be obtained from a variety of renewable sources and may be used in preparation of environment-friendly and renewable copolymers. By using the farnesene monomer, the mechanical stability of latex can be improved, and at the same time, the stickiness characteristics and durability of the final formed article can be improved.

The content of the farnesene monomer may be 1 to 39% by weight based on the total weight of the monomer mixture. For example, it may be 1% by weight, 5% by weight, 10% by weight, 15% by weight, 20% by weight, 25% by weight, 30% by weight, 35% by weight, 39% by weight, or a value between two of these values. When the content of the farnesene monomer is less than the range described above, the mechanical stability of latex may decrease or the stickiness of the final formed article may increase, and the durability thereof may decrease; and when the range described above is exceeded, the gel content of latex may excessively increase or the tensile strength and elongation of the final formed article may decrease.

The ethylenically unsaturated nitrile monomer may be one selected from the group consisting of acrylonitrile, methacrylonitrile, fumaronitrile, α-chloroacrylonitrile, α-cyano ethyl acrylonitrile, and a combination of two or more thereof, and for example, it may be acrylonitrile, but is not limited thereto.

The content of the ethylenically unsaturated nitrile monomer may be 1 to 50% by weight based on the total weight of the monomer mixture. For example, it may be 1% by weight, 5% by weight, 10% by weight, 15% by weight, 20% by weight, 25% by weight, 30% by weight, 35% by weight, 40% by weight, 45% by weight, 50% by weight, or a value between two of these values. When the content of the ethylenically unsaturated nitrile monomer is outside the range described above, the formed article may become excessively soft or excessively hard to reduce the wearing comfort of a user of the dip-formed article, or the oil resistance of the dip-formed article may decrease, and tensile strength may be lowered.

The ethylenically unsaturated acid monomer may be one selected from the group consisting of methacrylic acid, acrylic acid, itaconic acid, maleic acid, fumaric acid, maleic anhydride, citraconic anhydride, styrene sulfonate, monobutyl fumarate, monobutyl maleate, and mono-2-hydroxypropyl maleate, and a combination of two or more thereof, and for example, it may be methacrylic acid, but is not limited thereto.

The content of the ethylenically unsaturated acid monomer may be 1 to 10% by weight based on the total weight of the monomer mixture. For example, it may be 1% by weight, 2% by weight, 3% by weight, 4% by weight, 5% by weight, 6% by weight, 7% by weight, 8% by weight, 9% by weight, 10% by weight, or a value between two of these values. When the content of the ethylenically unsaturated acid monomer is outside the range described above, the formed article may be excessively soft or excessively hard to reduce the wearing comfort of a user of the dip-formed article, or the oil resistance of the dip-formed article may decrease, and tensile strength may be lowered.

Step (b) above is a step of adding an additive including an emulsifier and an inorganic solvent to the monomer mixture to prepare for emulsion polymerization.

The emulsifier may be an ionic surfactant, but is not limited thereto. For example, the emulsifier may be one selected from the group consisting of anionic surfactants, cationic surfactants, amphoteric surfactants, and a combination of two or more thereof.

The emulsifier may be one selected from the group consisting of alkylbenzene sulfonate, alkyldiphenyloxide disulfonate, aliphatic sulfonate, sulfuric acid ester salts of higher alcohols, α-olefin sulfonate, alkyl ether sulfuric acid ester salts, and a combination of two or more thereof, and for example, it may be sodium alkylbenzenesulfonate, but is not limited thereto.

The content of the emulsifier may be 0.1 to 5 parts by weight based on 100 parts by weight of the monomer mixture.

The inorganic solvent may be water, and for example, it may be ion exchange water, but is not limited thereto.

The additive may further include a molecular weight regulator.

The molecular weight regulator may be one selected from the group consisting of mercaptans such as an α-methylstyrene dimer, t-dodecyl mercaptan, n-dodecyl mercaptan, and octyl mercaptan; halogenated hydrocarbons such as carbon tetrachloride, methylene chloride, and methylene bromide; sulfur-containing compounds such as tetraethyl thiuram disulfide, dipentamethylene thiuram disulfide, and diisopropyl xanthogen disulfide; and a combination of two or more thereof, and for example, it may be t-dodecyl mercaptan, but is not limited thereto.

The content of the molecular weight regulator may be 0.2 to 0.65 parts by weight based on 100 parts by weight of the monomer mixture. For example, the content of the molecular weight regulator may be 0.2 parts by weight, 0.21 parts by weight, 0.22 parts by weight, 0.23 parts by weight, 0.24 parts by weight, 0.25 parts by weight, 0.26 parts by weight, 0.27 parts by weight, 0.28 parts by weight, 0.29 parts by weight, 0.3 parts by weight, 0.31 parts by weight, 0.32 parts by weight, 0.33 parts by weight, 0.34 parts by weight, 0.35 parts by weight, 0.36 parts by weight, 0.37 parts by weight, 0.38 parts by weight, 0.39 parts by weight, 0.4 parts by weight, 0.41 parts by weight, 0.42 parts by weight, 0.43 parts by weight, 0.44 parts by weight, 0.45 parts by weight, 0.46 parts by weight, 0.47 parts by weight, 0.48 parts by weight, 0.49 parts by weight, 0.5 parts by weight, 0.51 parts by weight, 0.52 parts by weight, 0.53 parts by weight, 0.54 parts by weight, 0.55 parts by weight, 0.56 parts by weight, 0.57 parts by weight, 0.58 parts by weight, 0.59 parts by weight, 0.6 parts by weight, 0.61 parts by weight, 0.62 parts by weight, 0.63 parts by weight, 0.64 parts by weight, 0.65 part, or a value between two of these values based on 100 parts by weight of the monomer mixture. When the content of the molecular weight regulator is less than the range described above, the gel content of latex may increase; and when the range described above is exceeded, the polymerization reaction rate may increase and thus the mechanical stability of latex may be decrease or the latex molecular weight may decrease and thus the tensile strength and durability of the final formed article may decrease.

Step (c) above is a step of emulsion-polymerizing the monomer mixture to prepare a copolymer latex, and the copolymer latex prepared through polymerization has a solid concentration of 30 to 60%, for example, 30%, 32%, 34%, 36%, 38%, 40%, 42%, 44%, 46%, 48%, 50%, 52%, 54%, 56%, 58%, 60%, or a value between two of these values, and the pH may be adjusted to 7-12, for example, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0 or a value between two of these values, but is not limited to thereto.

The polymerization in step (c) above may be performed at 10 to 90° C., for example, 10° C., 15° C., 20° C., 25° C., 30° C., 35° C., 40° C., 45° C., 50° C., 55° C., 60° C., 65° C., 70° C., 75° C., 80° C., 85° C., 90° C. or a temperature between two of these temperatures.

Step (c) above may include: adding a polymerization initiator to polymerize the monomer mixture; adding a polymerization terminator to stop the polymerization reaction; and removing unreacted monomers and adjusting the solid concentration and pH to obtain a copolymer latex.

The polymerization initiator may be one selected from the group consisting of inorganic peroxides such as sodium persulfate, potassium persulfate, ammonium persulfate, potassium perphosphate, and hydrogen peroxide; organic peroxides such as t-butyl peroxide, cumene hydroperoxide, p-menthane hydroperoxide, di-t-butyl peroxide, t-butylcumyl peroxide, acetyl peroxide, isobutyl peroxide, octanoyl peroxide, dibenzoyl peroxide, 3,5,5-trimethylhexanoyl peroxide, and t-butylperoxy isobutyrate; azobisisobutyronitrile, azobis-2,4-dimethyl valeronitrile, azobiscyclohexanecarbonitrile, methyl azobisisobutyrate, and a combination of two or more thereof, and for example, it may be potassium persulfate (potassium peroxodisulfate), but it is not limited thereto. The polymerization initiator content may be 0.02 to 1.5 parts by weight based on 100 parts by weight of the monomer mixture.

The polymerization terminator may be one that is added when the conversion rate of the polymerization reaction is 90% or more. For example, the polymerization terminator may one that is added when the conversion rate of the polymerization reaction is 90%, 91%, 92%, 93%, 94%, 95%, 96%, 97%, 98%, 99%, 99.9%, or a value between two of these values.

The polymerization terminator may be one selected from the group consisting of hydroxyl amines, hydroxy amine sulfates, diethyl hydroxy amines, hydroxy amine sulfonic acids, and alkali metal ions thereof; sodium hydroxide; sodium dimethyldithiocarbamate; aromatic hydroxy dithiocarboxylic acids such as hydroquinone derivatives, hydroxy diethyl benzene dithiocarboxylic acid, and hydroxy dibutyl benzene dithiocarboxylic acid; and a combination of two or more thereof, and for example, it may be sodium hydroxide, but is not limited thereto. The polymerization terminator content may be 0.02 to 1.5 parts by weight based on 100 parts by weight of the monomer mixture.

The solid concentration and pH of the copolymer latex may be regulated by adding additives such as pH adjusters, antioxidants, and antifoaming agents.

The pH adjuster may be an aqueous potassium hydroxide solution or ammonia water, but is not limited thereto.

The copolymer latex has excellent mechanical stability and thus can be applied to the preparation of a latex composition for stable dip-forming.

The gel content of the copolymer latex prepared in step (c) above may be 80% or less. For example, the gel content of the copolymer latex may be 80% or less, 75% or less, 70% or less, 65% or less, 60% or less, 55% or less, or 50% or less. The gel content of copolymer latex indicates the degree of crosslinking within the latex. A higher gel content means more crosslinking. When the gel content of copolymer latex exceeds the range described above, fine cracks or holes may be generated in the gloves, resulting in a decrease in tensile strength and elongation.

The method for preparing a latex composition for dip-forming may further include (d) adding a vulcanizing agent, a crosslinking agent, and a vulcanization accelerator to the copolymer latex to prepare a latex composition for dip-forming.

The vulcanizing agent may include sulfur such as powdered sulfur, precipitated sulfur, colloidal sulfur, surface-treated sulfur, and insoluble sulfur, but is not limited thereto. The vulcanizing agent attacks pi bonds in conjugated double bonds and crosslinks between polymer chains so that it may not only provide elasticity to a copolymer but also improve the chemical resistance of a dip-formed article. The content of the vulcanizing agent may be 0.1 to 3.0 parts by weight based on 100 parts by weight of the copolymer latex.

The crosslinking agent may be zinc oxide, but is not limited thereto. The content of the crosslinking agent may be 0.1 to 3.0 parts by weight based on 100 parts by weight of the copolymer latex.

The vulcanization accelerator may be one selected from the group consisting of 2-mercaptobenzothiazole, 2,2-dithiobisbenzothiazole-2-sulfenamide, N-cyclohexylbenzothiazole-2-sulfenamide, 2-morpholinothiobenzothiazole, tetramethylthiuram monosulfide, tetramethylthiuram disulfide, zinc diethyldithiocarbamate, zinc dibutyldithiocarbamate, diphenyl guanidine, di-o-tolylguanidine, and a combination of two or more thereof, and for example, it may be zinc dibutyldithiocarbamate, but is not limited thereto. The content of the vulcanization accelerator may be 0.1 to 3.0 parts by weight based on 100 parts by weight of the copolymer latex.

### Latex Composition for Dip-Forming

A latex composition for dip-forming according to another aspect of the present specification may be prepared by the method for preparing the latex composition for dip-forming described above, but is not limited thereto.

The latex composition for dip-forming may have a solid concentration of 10 to 30% and a pH of 9 to 11, but is not limited thereto.

### Dip-Formed Article

A dip-formed article according to still another aspect of the present specification is manufactured by dip forming the latex composition for dip-forming described above.

The dip-formed article has excellent mechanical properties such as tensile strength and elongation, excellent durability, low stickiness due to a high kinetic friction coefficient, and excellent quality such as tactile sensation, and thus may be applied to dip-formed articles of various fields.

The dip-formed article may be one selected from the group consisting of medical gloves, gloves for agricultural and livestock product processing, and industrial gloves, but is not limited thereto.

Hereinafter, examples of the present specification will be described in more detail. However, the following experimental results describe only representative experimental results among the examples, and the scope and content of the present invention may not be construed as being reduced or limited by the examples. Each effect of the various embodiments of the present invention not explicitly presented below will be specifically described in the corresponding section.

### Example 1

A 1 L high-pressure reactor equipped with a stirrer, a thermometer, a cooler, and a nitrogen gas inlet and configured to continuously introduce each component was prepared. After replacing the reactor atmosphere with nitrogen, a monomer mixture prepared by mixing 57% by weight of 1,3-butadiene, 10% by weight of β-farnesene monomers, 27% by weight of acrylonitrile, and 6% by weight of methacrylic acid based on the total weight of the monomer mixture was added. Afterwards, based on 100 parts by weight of the monomer mixture, 0.48 parts by weight of t-dodecyl mercaptan (TDDM) as a molecular weight regulator, 2 parts by weight of sodium alkylbenzenesulfonate as an emulsifier, and 120 parts by weight of ion exchange water were added to the reactor. After raising the temperature of the reactor to about 30° C., 0.3 parts by weight of potassium persulfate was added as a polymerization initiator.

At the time when the conversion rate reached about 95% based on the monomer mixture, 0.9 parts by weight of sodium hydroxide was added to stop the polymerization reaction. Afterwards, unreacted monomers were removed through a deodorizing process, and ammonia water, an antioxidant, an antifoaming agent, etc. were added to obtain a carboxylic acid-modified nitrile-based copolymer latex with a solid concentration of 45% by weight and a pH of 8.5.

### Example 2

A carboxylic acid-modified nitrile-based copolymer latex was obtained in the same manner as in Example 1 above except that a monomer mixture with 1,3-butadiene and 0-farnesene monomer contents of 52% by weight and 15% by weight, respectively, was added, and 0.52 parts by weight of t-dodecyl mercaptan was added.

### Example 3

A carboxylic acid-modified nitrile-based copolymer latex was obtained in the same manner as in Example 1 above except that a monomer mixture with 1,3-butadiene and 0-farnesene monomer contents of 47% by weight and 20% by weight, respectively, was added, and 0.60 parts by weight of t-dodecyl mercaptan was added.

### Example 4

A carboxylic acid-modified nitrile-based copolymer latex was obtained in the same manner as in Example 1 above except that a monomer mixture with 1,3-butadiene and 0-farnesene monomer contents of 37% by weight and 30% by weight, respectively, was added, and 0.64 parts by weight of t-dodecyl mercaptan was added.

### Comparative Example 1

A carboxylic acid-modified nitrile-based copolymer latex was obtained in the same manner as in Example 1 above except that a monomer mixture with a 1,3-butadiene content of 67% by weight without including a β-farnesene monomer was added.

### Comparative Example 2

A carboxylic acid-modified nitrile-based copolymer latex was obtained in the same manner as in Example 1 above except that a monomer mixture with 1,3-butadiene and β-farnesene monomer contents of 27% by weight and 40% by weight, respectively, was added

### Comparative Example 3

A carboxylic acid-modified nitrile-based copolymer latex was obtained in the same manner as in Example 1 above except that a monomer mixture with a β-farnesene monomer content of 67% by weight without including 1,3-butadiene was added.

### Comparative Example 4

A carboxylic acid-modified nitrile-based copolymer latex was obtained in the same manner as in Comparative Example 1 above except that 0.64 parts by weight of t-dodecyl mercaptan was added.

### Comparative Example 5

A carboxylic acid-modified nitrile-based copolymer latex was obtained in the same manner as in Example 1 above except that 0.68 parts by weight of t-dodecyl mercaptan was added.

### Comparative Example 6

A carboxylic acid-modified nitrile-based copolymer latex was obtained in the same manner as in Example 3 above except that 0.72 parts by weight of t-dodecyl mercaptan was added.

### Comparative Example 7

A carboxylic acid-modified nitrile-based copolymer latex was obtained in the same manner as in Example 4 above except that 0.76 parts by weight of t-dodecyl mercaptan was added.

Table 1 below summarizes the 1,3-butadiene content, the β-farnesene monomer content, the weight ratio of β-farnesene monomer to the total weight of 1,3-butadiene and β-farnesene monomer, and the content of t-dodecyl mercaptan, which is a molecular weight regulator, in the monomer mixture used in the polymerization of the carboxylic acid-modified nitrile-based copolymer latex of Examples 1 to 4 and Comparative Examples 1 to 7 above.

TABLE 1

| | | | | t-dodecyl |
|---|---|---|---|---|
| | | β-farnesene | β-farnesene | mercaptan |
| | 1,3-butadiene | monomer | monomer | content |
| | content | content | weight ratio | (parts |
| Item | (% by weight) | (% by weight) | (%) | by weight) |
| Example 1 | 57 | 10 | 15 | 0.48 |
| Example 2 | 52 | 15 | 22 | 0.52 |
| Example 3 | 47 | 20 | 30 | 0.60 |
| Example 4 | 37 | 30 | 45 | 0.64 |
| Comparative | 67 | 0 | 0 | 0.48 |
| Example 1 | | | | |
| Comparative | 27 | 40 | 60 | 0.48 |
| Example 2 | | | | |
| Comparative | 0 | 67 | 100 | 0.48 |
| Example 3 | | | | |
| Comparative | 67 | 0 | 0 | 0.64 |
| Example 4 | | | | |
| Comparative | 57 | 10 | 15 | 0.68 |
| Example 5 | | | | |
| Comparative | 47 | 20 | 30 | 0.72 |
| Example 6 | | | | |
| Comparative | 37 | 30 | 45 | 0.76 |
| Example 7 | | | | |

* β-farnesene monomer weight ratio (%) = [β-farnesene monomer content/(1,3-butadiene content + β-farnesene monomer content)] × 100

### Experimental Example 1

### Experimental Example 1-1: Mechanical Stability Evaluation

To evaluate the mechanical stability of each carboxylic acid-modified nitrile-based copolymer latex prepared according to Examples 1 to 4 and Comparative Examples 1 to 7 above, a latex stability test (Maron test) was performed. For the latex stability test, using a Maron tester, 30 g of a latex sample was placed in a sample frame and shear stress was applied at 49 N and 1000 rpm for 1 minute, and then the weight of the resulting aggregate was measured. The lower the numerical value of the weight of the aggregate obtained during the latex stability test, the better the evaluation of mechanical stability.

### Experimental Example 1-2: Gel Content Measurement

5 g of each carboxylic acid-modified nitrile-based copolymer latex prepared according to Examples 1 to 4 and Comparative Examples 1 to 7 above was added to 200 mL of isopropyl alcohol while stirring and coagulated. The coagulum was filtered through a 120-mesh gold mesh, dried in a constant temperature vacuum dryer at 50±2° C. and 750±10 mmHg for 1 hour, and then left in a desiccator until room temperature was reached. 0.25-0.35 g of the dried sample was accurately weighed (Wi) with a precision of 0.1 mg and placed in an Erlenmeyer flask, and 100 mL of methyl ethyl ketone (MEK) was added and stirred for 2 hours. Afterwards, the entire sample was filtered with filter paper. 20 mL of the filtrate was heated to vaporize methyl ethyl ketone, cooled to room temperature in a desiccator, and then accurately weighed (W_{f}) with a precision of 0.1 mg. The gel content in the sample was measured according to Equation 1 below, and an average value of two values were calculated to one decimal place.

$$Gel content ( \% ) = 100 - \frac{{w}_{f} \times 5}{{w}_{i}} \times 100 \qquad 〈 Equation 1 〉$$

Table 2 below shows the results of the mechanical stability evaluation and gel content measurement for each carboxylic acid-modified nitrile-based copolymer latex prepared according to Examples 1 to 4 and Comparative Examples 1 to 7.

TABLE 2

| Item | Coagulation weight (ppm) | Gel content (%) |
|---|---|---|
| Example 1 | 3,950 | 48.5 |
| Example 2 | 2,510 | 58.3 |
| Example 3 | 2,890 | 56.2 |
| Example 4 | 560 | 59.4 |
| Comparative Example 1 | 5,910 | 49.9 |
| Comparative Example 2 | 610 | 81.6 |
| Comparative Example 3 | 120 | 96 |
| Comparative Example 4 | 4,920 | 3.4 |
| Comparative Example 5 | 4,220 | 2.2 |
| Comparative Example 6 | 4,620 | 9.3 |
| Comparative Example 7 | 5,040 | 10.3 |

Referring to Table 2 above, the carboxylic acid-modified nitrile-based copolymer latex of Examples 1 to 4, in which the weight ratio of β-farnesene monomer to the total weight of 1,3-butadiene and β-farnesene monomer used in polymerization was 15%, 22%, 30%, and 45%, respectively, and the content of t-dodecyl mercaptan, which is a molecular weight regulator, was 0.48 to 0.64 parts by weight based on 100 parts by weight of the monomer mixture, exhibited better mechanical stability than Comparative Examples 1 and 4, in which a β-farnesene monomer was not used, and Comparative Examples 5 to 7, in which 0.68 to 0.76 parts by weight of a molecular weight regulator was added.

In addition, the gel content of the carboxylic acid-modified nitrile-based copolymer latex of Examples 1 to 4 was all excellent as 80% or less, but the gel content of the carboxylic acid-modified nitrile-based copolymer latex of Comparative Examples 2 and 3, in which the weight ratio of β-farnesene monomer to the total weight of 1,3-butadiene and β-farnesene monomer used in the polymerization was 60% and 100%, respectively, exceeded 80%.

### Manufacturing Example

Based on 100 parts by weight of each carboxylic acid-modified nitrile-based copolymer latex prepared according to Examples 1 to 4 and Comparative Examples 1 to 7 above, 1.0 part by weight of sulfur, 1.2 parts by weight of zinc oxide, and 0.5 parts by weight of zinc dibutyldithiocarbamate (ZDBC) as a vulcanization accelerator were added, and a 4% potassium hydroxide aqueous solution and double distilled water were further added to prepare a latex composition for dip-forming with a solid concentration of 18% and a pH of 10.0.

### Experimental Example 2

### Experimental Example 2-1: Evaluation of Mechanical Properties

Dumbbell-shaped specimens were produced from each latex composition for dip-forming prepared according to the Manufacturing Example above in accordance with ASTM D-412. A Universal Testing Machine (UTM) was used to pull the specimens at a stretching rate of 500 mm/min, and the tensile strength and elongation at break were measured to evaluate mechanical properties. Generally, the higher the numerical values of the tensile strength and elongation of a latex-formed article, the better the evaluation of dip quality.

### Experimental Example 2-2: Kinetic Friction Coefficient Measurement

After producing film specimens of each latex composition for dip-forming prepared according to the Manufacturing Example above, a friction coefficient meter was used to measure the friction coefficient of the latex side (the part that is in contact with the hand when manufacturing gloves) of the specimens. The size of the sled of the friction coefficient meter was set to 30 mm, and the vertical load was set to 100 g. After placing the sled on the specimen of the latex composition for dip-forming, the kinetic friction coefficient value was measured from the average value of the load values that were read while the sled was pulled 50 mm on the top surface of the film. The lower the kinetic friction coefficient, the more improved the evaluation of stickiness of the dip-formed article.

### Experimental Example 2-3: Durability Evaluation

From each latex composition for dip-forming prepared according to the Manufacturing Example above, specimens with a width of 30 mm, a length of 135 mm, and a thickness of 0.06 to 0.08 mm were produced. In a state of being stretched 20% in the longitudinal direction, the dip-formed article was immersed in a citric acid solution at 35° C. with a pH of 4.0 to 4.3 and fixed with a clamp. Then, a process in which the specimen was stretched for 10 seconds such that the longitudinal elongation became 50%, fixed for 2 seconds, and then relaxed for 10 seconds such that the longitudinal elongation became 20% was repeated using a motor so that the time taken to break the specimen was measured up to 180 minutes.

Table 3 below shows the results of the mechanical property evaluation, kinetic friction coefficient measurement, and durability evaluation of the specimens produced from each latex composition for dip-forming prepared according to the Manufacturing Example above.

TABLE 3

| | Tensile | | Kinetic friction | |
|---|---|---|---|---|
| | strength | Elongation | coefficient | Durability |
| Item | (MPa) | (%) | (μ) | (min) |
| Example 1 | 31.8 | 658 | 1.47 | 44.1 |
| Example 2 | 32.7 | 642 | Not measured | Not measured |
| Example 3 | 32.6 | 636 | 1.29 | 61.5 |
| Example 4 | 31.2 | 626 | 1.33 | >180 |
| Comparative | 33.8 | 687 | 1.67 | 26.9 |
| Example 1 | | | | |
| Comparative | 27.5 | 588 | Not measured | Not measured |
| Example 2 | | | | |
| Comparative | 4.7 | 131 | Not measured | Not measured |
| Example 3 | | | | |
| Comparative | 33.7 | 687 | Not measured | Not measured |
| Example 4 | | | | |
| Comparative | 27.7 | 685 | Not measured | Not measured |
| Example 5 | | | | |
| Comparative | 25.6 | 683 | Not measured | Not measured |
| Example 6 | | | | |
| Comparative | 23.8 | 647 | Not measured | Not measured |
| Example 7 | | | | |

Referring to Table 3 above, the carboxylic acid-modified nitrile-based copolymer latex of Examples 1 to 4, in which the weight ratio of β-farnesene monomer to the total weight of 1,3-butadiene and β-farnesene monomer used in polymerization was 15%, 22%, 30%, and 45%, respectively, and the content of t-dodecyl mercaptan, which is a molecular weight regulator, was 0.48 to 0.64 parts by weight based on 100 parts by weight of the monomer mixture, exhibited tensile strength at a level similar to the existing carboxylic acid-modified nitrile-based copolymer latex of Comparative Examples 1 and 4, in which a β-farnesene monomer was not used, indicating the mechanical properties required for latex gloves were satisfied. On the contrary, the tensile strength of the carboxylic acid-modified nitrile-based copolymer latex of Comparative Examples 2 and 3, in which the weight ratio of β-farnesene monomer was 60% and 100%, respectively, and the carboxylic acid-modified nitrile-based copolymer latex of Comparative Examples 5 to 7, in which 0.68 to 0.76 parts by weight of a molecular weight regulator was added, exhibited a level of less than 90% of the tensile strength of the carboxylic acid-modified nitrile-based copolymer latex of Comparative Example 1 or Comparative Example 4, in which a β-farnesene monomer was not used, confirming that mechanical properties were poor.

In addition, the elongation of the carboxylic acid-modified nitrile-based copolymer latex of Examples 1 to 4, in which the weight ratio of β-farnesene monomer to the total weight of 1,3-butadiene and β-farnesene monomer used in polymerization was 15%, 22%, 30%, and 45%, respectively, exhibited a level that was similar to the elongation of the existing carboxylic acid-modified nitrile-based copolymer latex of Comparative Examples 1 and 4, in which a β-farnesene monomer was not used, indicating the mechanical properties required for latex gloves were satisfied. On the contrary, the elongation of the carboxylic acid-modified nitrile-based copolymer latex of Comparative Examples 2 and 3, in which the weight ratio of β-farnesene monomer was 60% and 100%, respectively, exhibited a level of less than 90% of the elongation of the carboxylic acid-modified nitrile-based copolymer latex of Comparative Example 1, in which a β-farnesene monomer was not used, confirming that mechanical properties were poor.

In the case of kinetic friction coefficient and durability, the carboxylic acid-modified nitrile-based copolymer latex of Examples 1 to 4, in which a β-farnesene monomer was used, showed better values than those of the carboxylic acid-modified nitrile-based copolymer latex of Comparative Example 1, in which a β-farnesene monomer was not used.

### Advantageous Effects

The method for preparing a latex composition for dip-forming according to one aspect of the present specification can be applied to the preparation of a latex composition for dip-forming that is environment-friendly and has excellent mechanical stability by including a farnesene monomer.

In addition, the latex composition and the dip-formed article formed therefrom according to another aspect of the present specification have excellent mechanical properties, such as tensile strength and elongation, and durability and have improved stickiness, and thus can be applied to the manufacturing of medical gloves, gloves for agricultural and livestock product processing, and industrial gloves.

The effect of one aspect of the present specification is not limited to the effects described above and should be understood as including all effects that may be inferred from the configurations described in the detailed description or claims of the present specification.

The description of the present specification described above is for illustrative purposes, and it should be understood that those of ordinary skill in the art to which one aspect of the present specification belongs can easily modify it into other specific forms without changing the technical spirit or essential features described in the present specification. Therefore, it should be understood that the embodiments described above are illustrative in all respects and not restrictive. For example, each component described as a single type may be implemented in a distributed form, and likewise, components described as distributed may be implemented in a combined form.

The scope of the present specification is indicated by the following claims, and all changes or modifications derived from the meaning and scope of the claims and their equivalents should be construed as being included in the scope of the present specification.

What is claimed is:

1. A method for preparing a latex composition for dip-forming, the method comprising:
(a) preparing a monomer mixture including a conjugated diene-based monomer, a farnesene monomer, an ethylenically unsaturated nitrile monomer, and an ethylenically unsaturated acid monomer;
(b) adding an additive including an emulsifier and an inorganic solvent to the monomer mixture; and
(c) polymerizing the monomer mixture to prepare a copolymer latex,
wherein a weight ratio of the farnesene monomer to a total weight of the conjugated diene-based monomer and the farnesene monomer is 5 to 55%.

2. The method of claim 1, wherein the conjugated diene-based monomer is one selected from the group consisting of 1,3-butadiene, 2,3-dimethyl-1,3-butadiene, 2-ethyl-1,3-butadiene, 1,3-pentadiene, isoprene, and a combination of two or more thereof.

3. The method of claim 1, wherein the farnesene monomer is an α-farnesene monomer, a β-farnesene monomer, or a combination thereof.

4. The method of claim 1, wherein the ethylenically unsaturated nitrile monomer is one selected from the group consisting of acrylonitrile, methacrylonitrile, fumaronitrile, α-chloroacrylonitrile, α-cyano ethyl acrylonitrile, and a combination of two or more thereof.

5. The method of claim 1, wherein the ethylenically unsaturated acid monomer is one selected from the group consisting of methacrylic acid, acrylic acid, itaconic acid, maleic acid, fumaric acid, maleic anhydride, citraconic anhydride, styrene sulfonate, monobutyl fumarate, monobutyl maleate, mono-2-hydroxypropyl maleate, and a combination of two or more thereof.

6. The method of claim 1, wherein the additive further includes a molecular weight regulator.

7. The method of claim 6, wherein the molecular weight regulator is one selected from the group consisting of mercaptans such as an α-methylstyrene dimer, t-dodecyl mercaptan, n-dodecyl mercaptan, and octyl mercaptan; halogenated hydrocarbons such as carbon tetrachloride, methylene chloride, and methylene bromide; sulfur-containing compounds such as tetraethyl thiuram disulfide, dipentamethylene thiuram disulfide, and diisopropyl xanthogen disulfide; and a combination of two or more thereof.

8. The method of claim 6, wherein a content of the molecular weight regulator is 0.2 to 0.65 parts by weight based on 100 parts by weight of the monomer mixture.

9. The method of claim 1, wherein a gel content of the copolymer latex prepared in (c) above is 80% or less.

10. A latex composition for dip-forming, prepared by the method according to claim 1.

11. A dip-formed article formed by dip-forming the latex composition for dip-forming of claim 10.

12. The dip-formed article according to claim 11, wherein the dip-formed article is one selected from the group consisting of medical gloves, gloves for agricultural and livestock product processing, and industrial gloves.
